(19) United States
(12) Patent Application Publication
(10) Pub. No.: US 2019/0315821 A1
(43) Pub. Date: Oct. 17, 2019

(54) COMPOSITIONS COMPRISING HEPCIDIN AND METHODS OF USE THEREOF

(71) Applicant: La Jolla Pharmaceutical Company, San Diego, CA (US)

(72) Inventors: James Rolke, Poway, CA (US); Harry Wang, San Diego, CA (US)

(21) Appl. No.: 16/282,758

(22) Filed: Feb. 22, 2019

Related U.S. Application Data
(60) Provisional application No. 62/634,701, filed on Feb. 23, 2018.

(51) Int. Cl. C07K 14/47

(52) U.S. Cl. CPC C07K 14/4702

(57) ABSTRACT

Provided herein are improved compositions comprising hepcidin. In some aspects, provided herein are methods of treating a condition with a composition disclosed herein.

### CROSS-REFERENCES TO RELATED APPLICATIONS

This application claims the benefit of priority to U.S. Provisional Application No. 62/634,701, filed Feb. 23, 2018, which is incorporated herein by reference in its entirety and for all purposes.

### BACKGROUND

Iron is an essential element required for growth and survival of almost every organism. In mammals, the iron balance is primarily regulated at the level of duodenal absorption of dietary iron. Following absorption, ferric iron is loaded into apo-transferrin in the circulation and transported to the tissues, including erythroid precursors, where it is taken up by transferrin receptor-mediated endocytosis. Reticuloendothelial macrophages play a major role in the recycling of iron from the degradation of hemoglobin of senescent erythrocytes, while hepatocytes contain most of the iron stores of the organism in ferritin polymers.

In the case of iron deficiency, the pathophysiological consequences of gene defects identified are well understood because they usually result in loss of function of proteins directly involved in the pathway of iron absorption. The proteins include the iron transporters DMT1 (also called Nramp2 or DCT1), ferroportin (also called IREG1 or MTP1), and copper oxidases coupled to ferroportin, namely ceruloplasmin and haephastin. Additionally, several abnormalities associated with genetic iron overload have led to the identification of other proteins, but the functional role of these proteins remains poorly understood. In humans, hereditary hemochromatosis (HH) is a common autosomal recessive genetic disease caused by hyperabsorption of dietary iron leading to an iron overload in plasma and organs, including the pancreas, liver, and skin, resulting in damage caused by iron deposit.

Finally, although it has long been known that iron absorption is regulated in response to the level of body iron stores and to the amount of iron needed for erythropoiesis, the molecular nature of the signals that program the intestinal cells to adjust iron absorption remains unknown.

Hepcidin is the master regulator of extra-cellular iron in the body, controlling extracellular iron by the internalization and degradation of ferroportin. Because ferroportin is the primary mechanism by which iron is exported out of cells, the regulation of ferroportin allows control of iron in the plasma, extracellular space, and absorption of iron from the gut. As such, administration of hepcidin has the potential to be utilized for the treatment of acute and chronic iron excess. Nonclinical and clinical studies show that administration of hepcidin has a favorable safety profile and the iron lowering effects of a single subcutaneous injection can last up to 1 week. Improved hepcidin formulations would have the potential to benefit many patients.

### SUMMARY

Hepcidin is a 25 amino acid peptide containing eight cysteines, which form four disulfide bridges. This mature bioactive form of hepcidin derives from a precursor (pre-prohepcidin) of 84 amino acids which is subsequently processed into a 60- to 64-residue prohepcidin peptide and then finally into hepcidin 25 and secreted into the serum. The eight disulfide bridges form a compact hairpin structure. Under certain conditions, the disulfide bonds of hepcidin can break and/or rearrange to form improperly folded hepcidin (e.g., intramolecular disulfide bonding or aggregated hepcidin (intermolecular disulfide bonding). Aggregated hepcidin may include dimers (e.g. two molecules of hepcidin), trimers, oligomers and polymers of hepcidin (e.g. higher molecular weight aggregates). Depending on their size, aggregates may be soluble or insoluble. For pharmaceutical applications, aggregation is a concern as it can lead to reduced potency, reduced bioavailability, slower absorption, and/or increased immunogenicity and/or inflammation.

Provided herein are compositions of hepcidin suitable for pharmaceutical applications (e.g., pharmaceutical compositions). Also provided herein are methods of treating or preventing a condition in a subject with the compositions disclosed herein.

In some embodiments, the hepcidin is a purified hepcidin. In some embodiments, the purified hepcidin is substantially free of aggregated hepcidin. Aggregated hepcidin refers to two or more molecules of hepcidin which are covalently or ionically bound together. In some embodiments, aggregated hepcidin refers to two or more molecules of hepcidin which are covalently bound together. For example, aggregates can form due to disulfide scrambling with formation of improper inter-molecular disulfide bridges. Aggregated hepcidin may result in reduced potency of hepcidin and/or increased likelihood of an immunological response to administration of hepcidin due to production of antibodies to the aggregated material.

In preferred embodiments, the purified hepcidin is substantially free of thiol. Thiol can react with the disulfide bridges of hepcidin resulting in disulfide scrambling and potential oligomerization/polymerization of multiple hepcidin molecules to form aggregated hepcidin. Thiol can come from multiple sources including thiols left over from synthesis of the hepcidin, incomplete folding of hepcidin and breakage of disulfide bridges within folded hepcidin, improperly folded hepcidin and/or aggregated hepcidin.

In preferred embodiments, the purified hepcidin is substantially free of both thiol and aggregated hepcidin.

In some embodiments, the hepcidin is a salt of an acid or a base (e.g., hepcidin acetate). In preferred embodiments, salts of hepcidin include those in which the counter-ion is anionic in nature. Non-limiting examples include acetate, trifluoroacetate, chloride, bromide, citrate, sulfate, borate, lactate, maleate, malate, fumarate, phosphate, gluconate, uronate, succinate, propionate and/or mixtures thereof.

In some embodiments, the compositions provided herein comprise hepcidin and a thiol scavenger wherein the molar ratio of hepcidin to thiol scavenger is about 0.1:1 to about 10:1. In some embodiments, hepcidin (or a salt thereof) is dissolved in aqueous solution at a concentration of about 0.1 mg/mL to about 40 mg/mL. In preferred embodiments, the aqueous solution of hepcidin, or salt thereof, has a pH ranging from about 2 to about 6 (e.g., from about 3.5 to about 4.5). In some such embodiments, the hepcidin in aqueous solution further comprises a thiol scavenger (e.g., maleic acid) at a ratio of about 0.1:1 to about 10:1 (hepcidin to thiol scavenger).

In various embodiments, hepcidin is dissolved in a solvent, is suspended in a solvent, or is a colloid.

In some embodiments, the hepcidin is a hepcidin disclosed herein. In some embodiments, the hepcidin is human hepcidin. In some embodiments, the hepcidin comprises at least one of the amino acid sequences set forth in any one of SEQ ID NOs: 1 to 10.

Provided herein are methods of treating or preventing a condition in a subject by administering the pharmaceutical composition disclosed herein. The condition may be α-thalassemia, thalassemia intermedia, β-thalassemia, hemochromatosis, sickle cell disease, anemia, refractory anemia, hemolytic anemia, hepatocarcinoma, cardiomyopathy, diabetes, a viral infection, a bacterial infection, a fungal infection, or a protist infection. The compositions of the invention can be used in any condition in which a reduction of free or somatic iron is useful or desired.

### BRIEF DESCRIPTION OF DRAWINGS

FIG. 1 shows size exclusion chromatograms pre- and post-reduction of disulfide bonds, demonstrating that aggregation is a function of inter-molecular disulfide bonding.

FIG. 2 shows the effect of thiol on aggregate formation at pH 2.5 and pH 5.6.

FIG. 3 shows the effect of pH and temperature on aggregate formation.

FIG. 4 shows the effect of initial aggregate on future aggregate formation. Comparison of 9% starting aggregate (A) to 4% starting aggregate (B) indicates higher initial aggregation increases the rate of future aggregation.

FIG. 5 shows effect of thiol scavenger on aggregate formation. Aggregation of hepcidin at a concentration of 20 mg/mL was evaluated in the presence of maleic acid at 25° C. (A) and at 5° C. (B).

FIG. 6 shows the effect of salt form on aggregate formation. Different salt forms were evaluated in the absence (A) or presence (B) of maleic acid.

FIG. 7 shows the stability of an exemplary formulation of hepcidin-25 (SEQ ID NO: 1) at 25° C. and at 5° C.

FIG. 8 shows the effect of lower hepcidin concentration on stability. Hepcidin from two different lots were evaluated (one in each of A and B).

FIG. 9 shows the effect of formulated, purified hepcidin (with low aggregate and no detectable thiols) vs. unformulated, impure hepcidin on improving (A) potency and (B) drug bioavailability in human normal healthy volunteers as measured by % reduction in serum iron in healthy volunteers.

### DETAILED DESCRIPTION

Provided herein are compositions (e.g., pharmaceutical compositions) comprising a pharmaceutically acceptable form of purified hepcidin or a salt thereof. Also provided herein are compositions comprising a pharmaceutically acceptable salt of hepcidin in an aqueous solution (e.g., about 10 mg/mL of the hepcidin salt), preferably further comprising a thiol scavenger. In certain preferred embodiments, the thiol scavenger is maleic acid. In some such embodiments, the molar ratio of hepcidin to maleic acid is about 1:1, and preferably, wherein the aqueous composition has a pH of about 3.8-4.8.

In some aspects, provided herein are methods of treating or preventing a condition in a subject by administering to the subject a composition disclosed herein.

### Definitions

Throughout this specification, the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably. The terms “patient”, “subject” and “individual” are used interchangeably and refer to either a human or a non-human animal. These terms include mammals such as humans, primates, livestock animals (e.g., bovines, porcines, and equines), companion animals (e.g., canines, felines) and rodents (e.g., mice, rabbits and rats) and non-mammalian animals.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “administering” means providing a pharmaceutical agent or composition to a subject, and includes, but is not limited to, administering by a medical professional and self-administering. Such an agent, for example, may be hepcidin or a hepcidin analog.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient, for example, materials found in the United States Food and Drug Administration's Inactive Ingredients Database, and other non-toxic compatible substances employed in pharmaceutical formulations. In some embodiments, the composition comprises a pharmaceutically acceptable salt of hepcidin (e.g., a hepcidin disclosed herein) and a pharmaceutically acceptable carrier.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

In certain embodiments, agents of the invention may be used alone or conjointly administered with another type of therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour to about 12 hours, about 24 hours, about 36 hours, about 48 hours, about 72 hours, or about a week of one another. Thus, a subject who receives such treatment can benefit from a combined effect of different therapeutic agents.

“Treating” a disease in a subject or “treating” a subject having a disease refers to subjecting the subject to a pharmaceutical treatment, e.g., the administration of a drug, such that at least one symptom of the disease is decreased or prevented from worsening. The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, include prevention of an injury, pathology, condition, or disease. In various embodiments, the treatment or prevention may be complete (no detectable symptoms) or partial, such that fewer symptoms are observed than would likely occur absent treatment. In embodiments, treatment or prevention refers to a slowing the progression of the disease, disorder, or condition or inhibition progression thereof to a harmful or otherwise undesired state.

“Disease” or “condition” refers to a state of being or health status of a patient or subject capable of being treated with a composition or method provided herein.

Protein precursors such as prepropeptide and propeptides are used in accordance with their ordinary meaning in the art. For example, and without being bound to any particular theory or model, such may refer to an inactive peptide that can be turned into an active form by post-translational modification, such as breaking off or cleaving a portion, region, and/or domain of said peptide.

### Compositions

Whether a peptide (e.g., hepcidin) is produced by a recombinant or synthetic process, it may contain impurities resulting from degradation during product storage or from the method of producing the peptide. Peptide-related impurities include amino acid sequences related to, but different from, that of the active ingredient (e.g., SEQ ID NO:1), e.g., as a result of insertion, deletion, or other modifications (e.g., oxidation or glycosylation) to the amino acid sequence and/or residues of the peptide. Other impurities include diastereomers, hydrolysis products of labile amide bonds, residual solvents, reagents, and metals used during synthesis.

In certain preferred embodiments, the composition comprises a purified hepcidin. Depending on the method of manufacture, the hepcidin product may contain a number of impurities. For example, hepcidin prepared by solid phase peptide synthesis or liquid phase peptide synthesis may contain multiple impurities including, but not limited to, peptide chains that contain deletions (missing amino acids), insertions (additional amino acids), isomers (mis-ordered amino acids and various enantiomers) and other reaction by-products such as cleavage cocktails, cleavage scavengers, N-terminal protection groups, C-terminal protection groups, solid-phase resin linkers, and side-chain protecting groups. In addition, manufacture of hepcidin requires folding the peptide to form 4 disulfide bridges. Folding may result in isomers (improperly folded hepcidin) of the desired hepcidin and aggregates of hepcidin (two or more hepcidin molecules covalently bonded together via intermolecular disulfide bonding).

In preferred embodiments, the purified hepcidin composition refers to hepcidin peptide (e.g., SEQ ID NO:1) with 4 intramolecular disulfide bridges. In some such embodiments, purified hepcidin does not include residual solvents, reagents, and/or metals used during synthesis. In certain embodiments, purified hepcidin is at least about 80% free from other macromolecular components as measured by HPLC with detection at 214 nm. In preferred embodiments, purified hepcidin is at least about 90% free from other macromolecular components as measured by HPLC with detection at 214 nm. In some preferred embodiments, purified hepcidin is at least about 95% free from other macromolecular components as measured by HPLC with detection at 214 nm. In further preferred embodiments, purified hepcidin is at least about 96% free from other macromolecular components as measured by HPLC with detection at 214 nm. In more preferred embodiments, purified hepcidin is at least about 97% free from other macromolecular components as measured by HPLC with detection at 214 nm. In yet further preferred embodiments, purified hepcidin is at least about 98% free from other macromolecular components as measured by HPLC with detection at 214 nm. In the most preferred embodiments, purified hepcidin is at least about 99% free from other macromolecular components as measured by HPLC with detection at 214 nm.

In some embodiments, aggregated hepcidin includes peptide fragments, peptide chains that contain deletions, insertions, isomers, or other reaction by-products. In certain preferred embodiments, the purified hepcidin does not include peptide fragments, peptide chains that contain deletions, insertions, isomers, or other reaction by-products. In certain embodiments, aggregated hepcidin refers to hepcidin or hepcidin fragments with interpeptide disulfide bridges (e.g., a disulfide bridge between a first hepcidin protein and a second hepcidin protein).

Aggregation can be measured by several different methods. In some embodiments, aggregation is measured by size exclusion high-performance liquid chromatography (SEC-HPLC). This method allows for the separation and quantification of hepcidin monomer from any aggregates including dimer, trimer and multimers. In other preferred embodiments, aggregation is measured by an isocratic method performed on an HPLC system comprised of a temperature controlled auto-sampler, a binary or quaternary pump, and an ultraviolet detector. For example, in some such embodiments, the mobile phase consists of 0.05% TFA in 50% acetonitrile and water and human hepcidin-25 (SEQ ID NO: 1) is dissolved in of 0.05% TFA to 1 mg/mL concentration. Five microliters of the solution are injected onto a Zenix-C SEC-80 column of 7.8×300 mm in dimension, 80 Å pore size and 3 μm particle size (Sepax Technologies, Newark, Del. 19711). A flow rate of 1 mL/min is used to elute the analyte from the column and deliver to the UV detector set at 214 nm. The entire run time is 13 min. Empower™ chromatography software or equivalent software is used to integrate each chromatography peak and calculate % peak area at each respective retention time.

In some embodiments, the compositions provided herein comprise purified hepcidin and is substantially free of aggregated hepcidin, wherein aggregated hepcidin consists of two or more molecules of hepcidin covalently or ionically bound together. Such aggregates may form due to disulfide scrambling with formation of improper inter-molecular disulfide bridges.

Aggregated hepcidin may result in reduced potency of hepcidin and/or increased likelihood of an immunological response to administration of hepcidin due to production of antibodies to the aggregated material. Thus, it is advantageous to have a hepcidin that is substantially free of aggregate (e.g., less than 10% of the total peptide weight) and wherein new aggregates of hepcidin do not readily form.

Without wishing to be bound by theory, the observed increase in potency and bioavailability of the compositions provided herein is not merely due to the reduction in aggregates in the composition itself, but is also due to a reduction in post-administration aggregate formation in vivo, which would otherwise be promoted by unstable and reactive hepcidin free thiol groups as the formulation disperses and the local pH adjusts towards neutral/physiological pH.

In some embodiments, the weight percent of aggregation is less than 10% of the total peptide weight. In some embodiments, the weight percent of aggregation is less than 9% of the total peptide weight. In some preferred embodiments, the weight percent of aggregation is less than 8% of the total peptide weight. In further embodiments, the weight percent of aggregation is less than 7% of the total peptide weight. In some embodiments, the weight percent of aggregation is less than 6% of the total peptide weight. In preferred embodiments, the weight percent of aggregation is less than 5% of the total peptide weight. In certain embodiments, the weight percent of aggregation is less than 4% of the total peptide weight. In some such embodiments, the weight percent of aggregation is less than 3% of the total peptide weight. In some preferred embodiments, the weight percent of aggregation is less than 2% of the total peptide weight. In the most preferred embodiments, the weight percent of aggregation is less than 1% of the total peptide weight.

In some embodiments, the composition comprises purified hepcidin and is substantially free of thiol (e.g., unreacted, free sulfhydryl-containing cysteines). Thiols can react with the disulfide bridges of hepcidin resulting in disulfide scrambling and potential polymerization of multiple hepcidin molecules to form aggregated hepcidin. Such thiols can come from multiple sources, some of which include thiols left over from synthesis of the hepcidin, incomplete folding of hepcidin and breakage of disulfide bridges within folded hepcidin, improperly folded hepcidin and/or aggregated hepcidin. Thiols may be measured by several methods, which may include a thiol quantification assay kit such as that from Molecular Probes: Thiol and Sulfide Quantification Kit (T-6060). Such a kit uses cysteine as a standard so that thiols are reported as cysteine equivalents. In some such embodiments the thiol content is less than 0.20 mole thiol/mole hepcidin. In some preferred embodiments, the thiol content is less than 0.1 mole thiol/mole hepcidin. In more preferred embodiments, the thiol content is less than 0.05 mole thiol/mole hepcidin. In further preferred embodiments, the thiol content is less than 0.015 mole thiol/mole hepcidin. In the most preferred embodiments, the thiol content is less than 0.01 mole thiol/mole hepcidin.

In some embodiments, the compositions provided herein comprise purified hepcidin and are substantially free of both thiol and aggregated hepcidin.

In some embodiments, the hepcidin is a salt of an acid or a base (e.g., hepcidin acetate). In some such embodiments, hepcidin is a salt of an acid (e.g., those in which the counter-ion is anionic in nature). Non-limiting examples include acetate, trifluoroacetate, chloride, bromide, citrate, sulfate, borate, lactate, maleate, malate, fumarate, phosphate, diphosphate, gluconate, uronate, succinate, propionate, tartrate, nitrate, mesylate, calcium, potassium and/or mixtures thereof. In preferred embodiments, hepcidin is a salt of acetic acid (e.g., hepcidin acetate). In some embodiments, hepcidin is a salt of maleic acid (e.g., hepcidin maleate). In some embodiments, hepcidin is a mixture of salts of acetic acid and maleic acid (e.g., hepcidin acetate and hepcidin maleate).

In some embodiments, hepcidin is purified to be substantially free of both thiol and aggregated hepcidin, and comprises an anionic salt. In some such embodiments, hepcidin is purified to be substantially free of both thiol and aggregated hepcidin, and is a salt of acetic acid (hepcidin acetate). In some embodiments, hepcidin is purified to be substantially free of both thiol and aggregated hepcidin, and is a salt of trifluoroacetic acid (hepcidin trifluoroacetate). In some embodiments, hepcidin is purified to be substantially free of both thiol and aggregated hepcidin, and is a salt of hydrochloric acid (hepcidin chloride). In some embodiments, hepcidin is purified to be substantially free of both thiol and aggregated hepcidin, and is a salt of maleic acid (hepcidin maleate). In further embodiments, hepcidin is purified to be substantially free of both thiol and aggregated hepcidin, and is a mixture of salts of maleic acid (hepcidin maleate) and acetic acid (hepcidin acetate).

In some embodiments, the compositions provided herein comprise hepcidin and a thiol scavenger. Thiol scavengers, as used herein, are agents that react with thiols (e.g., an unreacted, free sulfhydryl, such as the thiol of a cysteine) to form an unreactive entity (e.g., via covalent bond formation) such that the new entity cannot react with other thiols or disulfide bonds. Thiol scavengers may also be agents that react with thiols to inhibit them from reacting with other thiols and/or disulfide bonds. Thiol scavengers prevent the scrambling of disulfide bridges and the formation of aggregates by trapping thiols that may form over time due to breakage of disulfide bonds within the hepcidin molecule or other hepcidin analogs that might be present.

In some embodiments, the thiol scavenger may be a molecule that can react with a thiol via an addition reaction, wherein the thiol is the nucleophile. Non-limiting examples of such thiol scavengers include N-ethylmaleimide (NEM) and other N-alkylmaleimides (e.g., N-methylmaleimide, N-propylmaleimide, or N-butylmaleimide), iodine, maleic acid, sodium maleate (and salts and esters thereof), fumaric acid (and salts and esters thereof), vinyl sulfones, acrylates, acrylamides, acrylonitriles, acrylonitriles, and methacrylates. In some embodiments, the thiol scavenger is a compound which includes an α,β-unsaturated carbonyl moiety.

In certain embodiments, the thiol scavenger may react with a thiol via a substitution reaction, wherein the thiol is the nucleophile. Such thiol scavengers include but are not limited to Thioanisole, 1,2-Ethanedithiol (EDT), 1,4-dithiothreitol (DTT), 1,4-dithioerythritol (DTE), 3,6-Dioxa-1,8-octane-dithiol (DODT), 1,2-Bis (2-mercaptoethoxy)ethane, 2,2′-(Ethylenedioxy)dimethanethiol, Methyl Sulfide, 2-mercaptoethanol, thiophenol, ethyl methyl sulfide, methionine, and cysteine.

In some embodiments, the thiol scavenger may be a molecule that reversibly inhibits a thiol from reacting with other thiols or disulfide bonds. In some such embodiments, the inhibitor is an acid with sufficient pKa to protonate the thiol, thereby making them very weak nucleophiles and reducing their reactivity. Non-limiting examples of such thiol scavengers include acetic acid, trifluoroacetic acid, hydrogen chloride, hydrogen bromide, citric acid, sulfuric acid, boric acid, lactic acid, maleic acid, malic acid, fumaric acid, formic acid, phosphoric acid, gluconic acid, uronic acid, succinic acid, propionic acid, tartric acid, nitric acid, mesylic acid, and/or mixtures thereof. In some embodiments, the molar ratio of hepcidin to thiol scavenger is about 0.1:1 to about 20:1. Preferably, the molar ratio of hepcidin to thiol scavenger is about 0.4:1 to about 5:1. More preferably, the molar ratio of hepcidin to thiol scavenger is about 0.7:1 to about 2:1. In some such embodiments, the molar ratio of hepcidin to thiol scavenger is about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 0.9:1, about 0.8:1, about 0.7:1, about 0.6:1, about 0.5:1, about 0.4:1, about 0.3:1, about 0.2:1, or about 0.1:1 Preferably, the molar ratio of hepcidin to thiol scavenger is about 0.7:1. In some embodiments the molar ratio of hepcidin to thiol scavenger is about 0.8:1, about 0.9:1, about 1:1, about 1.1:1, about 1.2:1, about 1.3:1, about 1.4:1, about 1.5:1, or about 1.6:1.

In some embodiments, the ratio of hepcidin to thiol scavenger is a concentration ratio, wherein the thiol scavenger is present at a concentration relative to the concentration of hepcidin. For example and without limitation a concentration ratio of 20:1 may represent 20 mg/mL hepcidin to 1 mg/mL thiol scavenger. In some such embodiments, Preferably, the molar ratio of hepcidin to thiol scavenger is about 0.4:1 to about 5:1. More preferably, the molar ratio of hepcidin to thiol scavenger is about 0.7:1 to about 2:1. In some such embodiments, the concentrations of hepcidin and thiol scavenger have a ratio of about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 0.9:1, about 0.8:1, about 0.7:1, about 0.6:1, about 0.5:1, about 0.4:1, about 0.3:1, about 0.2:1, or about 0.1:1. Preferably, the concentrations of hepcidin and thiol scavenger have a ratio of about 0.7:1. In some embodiments the concentrations of hepcidin and thiol scavenger have a ratio of about 0.8:1, about 0.9:1, about 1:1, about 1.1:1, about 1.2:1, about 1.3:1, about 1.4:1, about 1.5:1, or about 1.6:1.

In some embodiments, the compositions provided herein comprise purified hepcidin and are substantially free of both thiol and aggregated hepcidin, further comprise a thiol scavenger. In some such embodiments, the thiol scavenger is maleic acid, a salt of maleic acid, maleic acid and a salt of maleic acid, or an N-alkylmaleimide, such as NEM.

In some preferred embodiments, the composition comprises purified hepcidin and is substantially free of both thiol and aggregated hepcidin, and a thiol scavenger, wherein said hepcidin is an anionic salt. In some such embodiments, hepcidin is a salt of acetic acid (hepcidin acetate). In other such embodiments, said hepcidin is a salt of maleic acid (hepcidin maleate). In yet other such embodiments, said hepcidin is a salt of both maleic acid and acetic acid (hepcidin maleate and hepcidin acetate).

In certain embodiments, the composition is at least 25% more potent upon administration to a subject than the same amount of an unpurified hepcidin composition (e.g., a hepcidin composition comprising greater than 0.02 average mole thiol/[mole hepcidin]). In certain embodiments, the composition is at least 25% more potent upon administration to a subject than the same amount of a comparable hepcidin composition having, prior to administration, at least one of i) greater than 3% of the hepcidin in aggregated form or ii) an average thiol content of greater than 0.015 mole thiol per mole hepcidin.

In some such embodiments, the composition results in at least 25% greater iron reduction upon administration to a subject than the same amount of a comparable hepcidin composition having, prior to administration, at least one of i) greater than 3% of the hepcidin in aggregated form or ii) an average thiol content of greater than 0.015 mole thiol per mole hepcidin.

In further embodiments, the composition has at least 25% longer duration of effect upon administration to a subject than the same amount of a comparable hepcidin composition having, prior to administration, at least one of i) greater than 3% of the hepcidin in aggregated form ii) an average thiol content of greater than 0.015 mole thiol per mole hepcidin.

It would be appreciated by one of skill in the art that an amount, as disclosed herein, can refer to a “concentration” or “molar” amount. Thus, a comparison of an amount of the composition of the invention to the same amount of a comparable hepcidin composition refers to the quantity of hepcidin in said amounts.

In some embodiments, the composition comprises hepcidin in an aqueous solution. Alternatively, the composition of hepcidin or purified hepcidin may be a powder or lyophilization product in a vial that when dissolved or suspended in an aqueous solution results in an aqueous composition as described herein.

In certain preferred embodiments, the composition comprises hepcidin (or a salt thereof, e.g., hepcidin acetate, hepcidin maleate, hepcidin chloride, hepcidin trifluoroacetate, or mixtures thereof) in an aqueous solution at a concentration of about 0.1 mg/mL to about 40 mg/mL. In preferred embodiments the concentration is about 1 mg/mL to about 20 mg/mL. In more preferred embodiments, the concentration of hepcidin is about 5 mg/mL to about 10 mg/mL. In some embodiments, the composition may comprise hepcidin (e.g., human hepcidin) or a salt thereof, dissolved in aqueous solution at a concentration of about 0.1 mg/mL, about 0.2 mg/m, about 0.3 mg/ml, about 0.4 mg/ml, about 0.5 mg/mL, about 0.6 mg/ml, about 0.7 mg/ml, about 0.8 mg/mL, about 0.9 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/ml, about 15 mg/mL, about 16 mg/mL, about 17 mg/mL, about 18 mg/mL, about 19 mg/mL about 20 mg/mL, about 25 mg/mL, about 30 mg/mL, about 35 mg/mL, about 40 mg/mL, about 50 mg/ml or about 100 mg/mL of hepcidin. In preferred embodiments, hepcidin or salts thereof, are dissolved in aqueous solution of about 5 mg/mL, about 10 mg/mL, about 15 mg/mL or about 20 mg/mL.

In preferred embodiments, the aqueous solution of hepcidin has an acidic pH (e.g., <pH 7). In some such embodiments, the aqueous solution of hepcidin has a pH ranging from about 2 to about 6, preferably from about 3 to about 5, from about 3.5 to about 4.5, or, most preferably, from about 3.8 to about 4.8. In certain embodiments, the pH is about 3.9. In some embodiments, the pH is about 4.0. In further embodiments, the pH is about 4.1. In yet other embodiments, the pH is about 4.2. In still other embodiments, the pH is about 4.3. In additional embodiments, the pH is about 4.4. In yet other embodiments, the pH is about 4.5. In further embodiments, the pH is about 4.6. In still other embodiments, the pH is about 4.7. In additional embodiments, the pH is about 4.8.

By way of non-limiting example and depending upon the ratio of hepcidin to thiol scavenger used, the concentration of hepcidin requires thiol scavenger at a concentration of about 0.01 mg/mL to about 10 mg/mL, inclusive. Preferably, the required concentration of thiol scavenger is about 0.1 mg/mL to about 1 mg/mL. In certain such embodiments, the concentration of thiol scavenger is about 0.1 mg/mL. In other embodiments, the concentration of thiol scavenger is about 0.2 mg/mL. In further embodiments, the concentration of thiol scavenger is about 0.3 mg/mL. In still other embodiments, the concentration of thiol scavenger is about 0.4 mg/mL. In additional embodiments, the concentration of thiol scavenger is about 0.5 mg/mL. In yet other embodiments, the concentration of thiol scavenger is about 0.6 mg/mL. In further embodiments, the concentration of thiol scavenger is about 0.7 mg/mL. In additional embodiments, the concentration of thiol scavenger is about 0.8 mg/mL. In still other embodiments, the concentration of thiol scavenger is about 0.9 mg/mL. In further embodiments, the concentration of thiol scavenger is about 1.0 mg/mL.

In some embodiments, the composition includes 20 mg/mL hepcidin acetate and 1 mg/mL of maleic acid. In some embodiments, the composition includes 10 mg/mL hepcidin acetate and 0.5 mg/mL of maleic acid.

In some embodiments, the composition comprises an aqueous solution of hepcidin, wherein the aqueous solution comprises purified hepcidin substantially free of thiols (e.g., as determined by a quantification method described herein). In some such embodiments the aqueous solution of hepcidin is substantially free of aggregate. In further embodiments, the aqueous solution of hepcidin is substantially free of thiols and aggregate. In some embodiments the hepcidin is a salt of an acid. In some embodiments, the aqueous solution further comprises a thiol scavenger. In some embodiments, the aqueous solution of hepcidin may be referred to as a formulation.

In some embodiments, hepcidin is dissolved in a solvent other than pure water. In some such embodiments, the preferred solvent is saline, e.g., 0.5%-1.5% saline, preferably about 0.9% saline. In other embodiments, the preferred solvent is acetonitrile.

In some embodiments, hepcidin is suspended in a solvent (e.g., saline).

In some embodiments, hepcidin is a colloid.

In some embodiments, the composition comprising hepcidin or purified hepcidin may be a powder or lyophilization product in a vial. The dry product, when dissolved or suspended in a solution, would achieve the same preferred concentrations and/or ratios as defined in this specification for solutions of hepcidin.

In some embodiments, hepcidin or purified hepcidin is formulated at a low pH (e.g., at a pH between about 1 and about 4) and used in conjunction with a second solution at the time of administration to adjust the pH closer to neutral pH (e.g., between pH about 4 and pH about 7). In some embodiments, the pH adjuster comprises an aqueous solution containing a buffer at a higher pH which when mixed with the lower pH solution (e.g., the hepcidin formulation) adjusts the final pH to a more neutral target (e.g., pH 4 to pH 7). Examples of suitable buffers include, but are not limited to, acetate, maleate, methanesulfonate, bicarbonate, carbonate, borate, citrate, lactate, phosphate or combinations thereof. In some embodiments, the volumes of solutions mixed may be the same or different. The volume of hepcidin solution to adjusting solution could be at least 0.5:1, at least 0.6:1, at least 0.7:1, at least 0.8:1, at least 0.9:1, at least 1:1, at least 2:1, at least 3:1, at least 4:1, at least 5:1, at least 6:1, at least 7:1, at least 8:1, at least 9:1, at least 10:1 or at least 20:1.

In other embodiments, the pH adjuster is an ion-exchange resin. Examples of said resins include, but are not limited to, Strong Cation Exchangers such as Sulfopropyl (SP), Sulfoethyl (SE), Sulfomethyl (S); Strong Cation Exchangers on Polystyrene such as Dowex (Sulfonic Acid); Weak Cation Exchangers on Polyacrylic such as Amberlite (Carboxylic Acid), and Dowex (Carboxylic Acid). Mixed Bed Resins, Chelating Resins such as Iminodiacetic Acid, Polymer Catalysts such as Amberlyst Strong Acid (Sulfonic Acid), Dowex Strong Acid (Sulfonic Acid), and Amberlyst Weak Base (Alkyl Amine). It should be recognized that other methods of pH adjustment could also be employed to adjust the pH closer to neutral.

In some embodiments, the compositions described herein are stored below room temperature (e.g., below 25° C.). In some embodiments, the compositions described herein are stored at about 25° C. In some embodiments, the compositions described herein are stored at about 5° C.

In some embodiments, the compositions described herein are stored at room temperature (e.g., approximately 25° C.). In preferred embodiments, compositions described herein are stored at lower temperatures, such as below 10° C., 5° C., or even below 0° C. (i.e., the composition may be frozen and thawed prior to administration). In some such embodiments, the composition is essentially stable when stored at or below 10° C. or 5° C., e.g., after storage for a year or even two years, the composition comprises at least 80% of the original concentration of monomeric hepcidin (e.g., less than 20% aggregate), preferably at least 90% of the original concentration of monomeric hepcidin (e.g., less than 10% aggregate), more preferably at least 95% of the original concentration of monomeric hepcidin (e.g., less than 5% aggregate), and most preferably at least 97% of the original concentration of monomeric hepcidin (e.g., less than 3% aggregate). In certain embodiments, the compositions described herein are stable (e.g., aggregate formation is less than or equal to 0.05% total hepcidin peptide weight) when stored at 5° C. for 3 years. In some embodiments, the compositions described herein are stable (e.g., aggregate formation is less than or equal to 0.05% total hepcidin peptide weight) when stored at 5° C. for 2 years. In further embodiments, the compositions described herein are stable (e.g., aggregate formation is less than or equal to 0.05% total hepcidin peptide weight) when stored at 5° C. for 1 year. In yet further embodiments, the compositions described herein are stable (e.g., aggregate formation is less than or equal to 0.05% total hepcidin peptide weight) when stored at 25° C. for 1 year.

Peptides in pharmaceutical compositions may adsorb onto vessels used in the manufacture and administration. The addition of anti-adsorbents known in the art may decrease the loss of peptides to adsorption. The composition may further comprise one or more anti-adsorbent, such as a surfactant, saccharide, amino acid and/or protein. The anti-adsorbent may be present at any concentration.

Another mode of hepcidin degradation is oxidation. Hepcidin contains methionine in that can undergo oxidation to form a sulfoxide. In order to inhibit oxidation of methionine residues, various antioxidants can be used. Accordingly, in some embodiments, the composition of hepcidin may further comprise one or more antioxidants. For example, antioxidants may be free-radical scavengers such as L-methionine, L-cysteine, L-histidine, L-cysteine, L-glycine, L-aspartic acid, or L-tryptophan. In some embodiments, the composition of purified hepcidin further includes methionine (e.g., L-methionine). Other exemplary antioxidants that may be used include ascorbic acid, pyridoxine, and trolox. Amino acids can be used at any suitable concentration, such as concentrations of 0-0.2%. EDTA chelating agent can also be used to prevent oxidation by divalent metals, such as in a concentration range of 0-0.2%. In certain embodiments, the ratio of hepcidin to methionine is 1:2, 1:3, 1:4, 1:5, 1:6, 1:7, 1:8, 1:9, or 1:10. In some such embodiments, the composition of purified hepcidin includes methionine at a 1:1 ratio of hepcidin to methionine. In some embodiments, the composition of purified hepcidin includes methionine at a 1:10 ratio of hepcidin to methionine.

In some embodiments, the compositions disclosed herein comprise one or more surfactant(s). In certain such embodiments, the compositions may comprise a combination of antioxidants and surfactants that can inhibit both aggregation and oxidation. Acetate buffer may be used in any suitable concentration, such as 10 mM, 20 mM, 40 mM, 100 mM or 100 mM. At these concentrations, a very strong effect of ionic strength is achievable. However, higher ionic strengths promote hepcidin degradation. Arginine together with methionine can be used in combination to inhibit both aggregation and oxidation. In such embodiments, arginine could be present at a concentration 0.1 N, 0.3 N, 0.6 N, or 1.0 N.

In certain aspects of the invention, provided herein are hepcidin proteins (e.g., SEQ ID NO:1) covalently bonded to a thiol scavenger (e.g., wherein the thiol scavenger reacted with a thiol of a cysteine of hepcidin). In some embodiments, a hepcidin protein is covalently bonded to maleic acid. In some such embodiments, the hepcidin covalently bonded to a thiol scavenger (e.g., maleic acid) is the product of a reaction between the hepcidin protein (e.g., SEQ ID NO:1) and a thiol scavenger. It will be understood that the covalently bonded hepcidin protein and thiol scavenger described herein are the remnants of the reactant hepcidin protein (e.g., SEQ ID NO:1, wherein at least one of the disulfide bonds of bioactive hepcidin is unformed or improperly formed) and a thiol scavenger, wherein each reactant now participates in the covalent bond between the hepcidin protein and the thiol scavenger. In certain embodiments, the invention provides compositions and/or formulations comprising properly folded hepcidin and hepcidin protein covalently bonded to maleic acid (e.g., at concentrations less than 10%, less than 5%, and preferably less than 2% or even less than 1% of the concentration of the properly folded hepcidin).

In further aspects, provided herein are methods of making purified hepcidin, the method including i) mixing a hepcidin molecule with a thiol scavenger (e.g., maleic acid) to form a hepcidin-thiol scavenger mixture, and ii) performing tangential flow filtration on the hepcidin-thiol scavenger mixture, to form purified hepcidin.

### Hepcidin

Hepcidin, as used herein, is a physiologically active peptide comprising any one of the amino acid sequences disclosed herein. The hepcidin peptide may be a 25-amino acid peptide with the amino acid sequence set forth in SEQ ID NO: 1. The hepcidin peptide may be a cleavage product of a larger pre-propeptide (e.g., the 84-mer peptide, such as the pre-propeptide encoded by the human hepcidin gene), which is further processed into a prohepcidin precursor (e.g., 60- to 64-mer peptide), which the cell membrane protein furin can convert into the hepcidin peptide. The term “hepcidin” as used herein may therefore refer to a peptide comprising the sequence set forth in SEQ ID NO: 1, including peptides that are longer than 25 amino acids, such as peptides consisting of 26 to 100 amino acids, or precursor peptides such as pre-prohepcidin or prohepcidin that can be converted to hepcidin or an analog having hepcidin activity.

Conservative amino acid substitutions, additions, and deletions may be made to SEQ ID NO: 1 without significantly affecting the function of hepcidin. Thus, the term “hepcidin” may refer to a peptide comprising an amino acid sequence having at least 90%, 91%, 92%, 93%, 94%, 95%, 96%, 97%, 98%, or 99% sequence homology with the amino acid sequence set forth in SEQ ID NO: 1. Sequence homology may be determined using any suitable sequence alignment program, such as Protein Blast (blastp) or Clustal (e.g., Clustal, ClustalW, ClustalX, or Clustal Omega), e.g., using default parameters, such as default weights for gap openings and gap extensions. Sequence homology may refer to sequence identity. The term “hepcidin” may refer to a peptide comprising an amino acid sequence that is identical to the sequence set forth in SEQ ID NO: 1 except that 1, 2, 3, 4, 5, 6, 7, 8, 9, or 10 amino acids of SEQ ID NO: 1 are substituted with different amino acids. In preferred embodiments, hepcidin comprises a cysteine at each of the positions in which a cysteine occurs in SEQ ID NO:1.

“Hepcidins” according to the invention can also include mini-hepcidins and hepcidin mimetic peptides, for example as disclosed in U.S. Pat. No. 8,435,941, hereby incorporated by reference, in particular for its disclosure of compounds that share one or more activities with hepcidin.

Further, the compositions and formulations contemplated herein are suitable for formulating modified and/or derivatized forms of hepcidin, such as, and without limitation, hepcidins bearing those modifications that may affect bio-availability, concentration, duration of action, and/or membrane permeability, such as pegylated hepcidins and other covalently-modified hepcidins.

SEQ ID NO: 1
DTHFPICIFCCGCCHRSKCGMCCKT

In some embodiments, the hepcidin is a hepcidin disclosed herein, such as human hepcidin. In some embodiments, the hepcidin may comprise the amino acid sequence of any one of SEQ ID NOs: 1-10 or analogs and/or mimetics thereof

N-terminal and C-terminal residues may be deleted from the hepcidin peptide without significantly affecting its function. Thus, in some embodiments, hepcidin refers to a peptide comprising the sequence set forth in SEQ ID NO: 2, SEQ ID NO: 3, or SEQ ID NO: 4, or a peptide comprising an amino acid sequence having at least 90%, 91%, 92%, 93%, 94%, 95%, 96%, 97%, 98%, or 99% sequence homology with the amino acid sequence set forth in SEQ ID NO:2, SEQ ID NO:3, SEQ ID NO:4, or SEQ ID NO:5. The term hepcidin may refer to a peptide comprising an amino acid sequence that is identical to the sequence set forth in SEQ ID NO:2, SEQ ID NO:3, SEQ ID NO:4, or SEQ ID NO:5 except that 1, 2, 3, 4, 5, 6, 7, 8, 9, or 10 amino acids of SEQ ID NO:2, SEQ ID NO:3, SEQ ID NO:4, or SEQ ID NO:5 are substituted with different amino acids. In some embodiments, hepcidin comprises a cysteine at each of the positions in which a cysteine occurs in SEQ ID NO:2, SEQ ID NO:3, SEQ ID NO:4, or SEQ ID NO:5.

SEQ ID NO: 2
PICIFCCGCCHRSKCGMCCKT
SEQ ID NO: 3
PICIFCCGCCHRSKCGMCC
SEQ ID NO: 4
ICIFCCGCCHRSKCGMCCKT
SEQ ID NO: 5
CIFCCGCCHRSKCGMCC

In some embodiments, the hepcidin refers to a peptide comprising an amino acid sequence that is identical to the sequence set forth in SEQ ID NO: 6, SEQ ID NO: 7, SEQ ID NO: 8, SEQ ID NO:9, or SEQ ID NO:10. In SEQ ID NO:6, SEQ ID NO:7, SEQ ID NO:8, SEQ ID NO: 9, or SEQ ID NO: 10, the amino acids labeled “X” may be any amino acid, including naturally occurring and non-naturally occurring amino acids. In some embodiments, each of the amino acids labeled “X” is a naturally occurring amino acid.

SEQ ID NO: 6
XXHXPXCXXCCGCCHRSKCGMCCXX
SEQ ID NO: 7
PXCXXCCGCCHRSKCGMCCKX
SEQ ID NO: 8
PXCXXCCGCCHRSKCGMCC
SEQ ID NO: 9
XCXXCCGCCHRXXCGXCCKX
SEQ ID NO: 10
CXXCCGCCHRXXCGXCC

In preferred embodiments, the hepcidin is a molecule that specifically binds to ferroportin and/or iron (e.g., an iron cation). The hepcidin may comprise 1, 2, 3, or 4 disulfide bonds. In preferred embodiments, hepcidin comprises four disulfide bonds, e.g., intramolecular disulfide bonds. In preferred embodiments, each of the eight cysteines of SEQ ID NOs: 1, 2, 3, 4, 5, 6, 7, 8, 9, or 10 participates in one of four intramolecular disulfide bonds with another one of the eight cysteines.

In preferred embodiments, the hepcidin has about 10% to 1000% of the activity of a 25 amino acid long peptide comprising the amino acid sequence set forth in SEQ ID NO: 1, i.e., wherein the 25 amino acid long peptide comprises the four intramolecular disulfide bonds found in native human hepcidin. For example, the hepcidin may have about 50% to about 200% of the activity of a 25 amino acid long peptide comprising the amino acid sequence set forth in SEQ ID NO:1 (i.e., wherein the 25 amino acid long peptide comprises the four intramolecular disulfide bonds found in native human hepcidin), such as about 75% to about 150% of the activity, about 80% to about 120% of the activity, about 90% to about 110% of the activity, or about 95% to about 105% of the activity. The term “activity” may refer to the ability of hepcidin to specifically bind to ferroportin, e.g., thereby inhibiting the transport of intracellular iron into the extracellular space, inhibiting the absorption of dietary iron, and/or reducing serum iron concentration. Activity may refer to the ability of hepcidin to inhibit the transport of intracellular iron into the extracellular space. Activity may refer to the ability of hepcidin to inhibit the absorption of dietary iron. Activity may refer to the ability of hepcidin to reduce serum iron concentration in vivo. Such molecules preferably retain at least 10% of one characteristic hepcidin activity, such as iron-lowering activity (hepcidin mimetic).

### Methods

Provided herein are methods of treating or preventing a condition in a subject by administering a composition (e.g., pharmaceutical composition) as disclosed herein. Various methods for administering hepcidin compositions are disclosed in, for example, published international application WO 2017/120419, WO 2018/048944, and WO 2018/118979, incorporated herein by reference. In some embodiments, the method is treating or preventing a condition in a subject by administering a composition (e.g., pharmaceutical composition) as disclosed herein at a therapeutically effective amount. In certain embodiments, the method is preventing a condition in a subject by administering a composition (e.g., pharmaceutical composition) as disclosed herein at a therapeutically effective amount. The condition may be α-thalassemia, thalassemia intermedia, β-thalassemia, hemochromatosis (e.g., hereditary hemochromatosis), sickle cell disease, anemia, refractory anemia, hemolytic anemia, hepatocarcinoma, cardiomyopathy, prevention of acute kidney injury due to transient overexposure to iron, diabetes, a viral infection, a bacterial infection, a fungal infection, or a protist infection.

In some aspects, provided herein are methods of treating a condition in a subject by administering a composition disclosed herein to the subject, e.g., in a therapeutically effective amount. The condition may be α-thalassemia, thalassemia intermedia, β-thalassemia, hemochromatosis, sickle cell disease, refractory anemia, or hemolytic anemia. The condition may be hemochromatosis and the hemochromatosis may be hereditary hemochromatosis. The condition may be hemochromatosis and the hemochromatosis may be associated with hepatocarcinoma, cardiomyopathy, or diabetes. The condition may be anemia. Anemia may be, for example, a hemoglobinopathy, sideroblastic anemia, anemia associated with myelodysplastic syndrome (MDS), or a congenital anemia. The condition may be myelodysplastic syndrome (MDS). The condition may be hemoglobinopathy, sideroblastic anemia, or a congenital anemia. In some embodiments, the condition may be hepatocarcinoma, cardiomyopathy, or diabetes.

The condition may be a viral, bacterial, fungal, or protist infection. In some embodiments, the condition is a bacterial infection, and the bacteria is Escherichia coli, Mycobacterium (such as M. africanum, M. avium, M. tuberculosis, M. bovis, M. canetti, M. kansasii, M. leprae, M. lepromatosis, or M. micron), Neisseria cinerea, Neisseria gonorrhoeae, Staphylococcus epidermidis, Staphylococcus aureus, or Streptococcus agalactiae. In some embodiments, the condition is a fungal infection, and the fungus is Candida albicans. In some embodiments, the condition is a protist infection, and the protist is Trypanosoma cruzi, Plasmodium (such as P. falciparum, P. vivax, P. ovale, or P. malariae), Trypanosoma brucei (such as T. brucei gambiense or T. brucei rhodesiense), or Leishmania. The condition may be a viral, bacterial, fungal, or protist infection, and the viral, bacterial, fungal, or protist infection may be resistant to one or more agents for treating the viral, bacterial, fungal, or protist infection. The condition may be a bacterial infection and the bacterial infection may be tuberculosis. The condition may be Chagas disease, malaria, African sleeping sickness, or leishmaniasis. In some embodiments, the condition is a viral infection, and the virus is hepatitis B, hepatitis C, or dengue virus.

### Subjects

The subject may be a mammal. The subject may be a rodent, lagomorph, feline, canine, porcine, ovine, bovine, equine, or primate. In preferred embodiments, the subject is a human. The subject may be a female or male. The subject may be an infant, child, or adult.

In some embodiments, administering the composition to a subject decreases the serum iron concentration of the subject. For example, administering the composition may decrease the serum iron concentration of a subject by at least about 5 μg/dL, at least about 10 μg/dL, at least about 5 μg/dL, at least about 20 μg/dL, at least about 30 μg/dL, at least about 40 μg/dL, at least about 50 μg/dL, at least about 60 μg/dL, at least about 70 μg/dL, at least about 80 μg/dL, at least about 90 μg/dL, or at least about 100 μg/dL. Administering the composition may decrease the serum iron concentration of the subject for at least 24 hours. For example, administering the composition may decrease the serum iron concentration of the subject by at least about 5 μg/dL for a period of time of at least 24 hours. Administering the composition may decrease the serum iron concentration of the subject by at least about 5 μg/dL for at least 4 hours, at least 6 hours, or at least 12 hours. Administering the composition may decrease the serum iron concentration of the subject by at least about 5 μg/dL for at least 1 day, at least 2 days, at least 3 days, at least 4 days, at least 5 days, at least 6 days, at least 7 days, or at least 8 days. Administering the composition may decrease the serum iron concentration of the subject by at least about 5%, such as at least about 10%, at least about 15%, at least about 20%, at least about 25%, or at least about 30%. Administering the composition may decrease the serum iron concentration of the subject by at least about 5% for at least 4 hours, at least 6 hours, or at least 12 hours. Administering the composition may decrease the serum iron concentration of the subject by at least about 5% for at least 1 day, at least 2 days, at least 3 days, at least 4 days, at least 5 days, at least 6 days, at least 7 days, or at least 8 days.

Representative methods of treating or preventing a condition in a subject using hepcidin compositions are disclosed in published international application WO 2017/120419, incorporated herein by reference.

### Routes of Administration

The compositions disclosed herein can be administered by a variety of means known in the art. In some aspects, the compositions of the invention are suitable for parenteral administration, which includes, e.g., intravenous, intramuscular, intra-arterial, intradermal, subcutaneous, intraperitoneal, intraventricular, and intracranial. Preferably, these compositions may be administered, for example, intraperitoneally, intravenously, internally, or intrathecally. In some aspects, the compositions are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

The composition may be administered topically, enterally, or parenterally. The composition may be administered subcutaneously, intravenously, intramuscularly, intranasally, by inhalation, orally, sublingually, by buccal administration, topically, transdermally, or transmucosally. The composition may be administered by injection. In preferred embodiments, the composition is administered by subcutaneous injection, orally, intranasally, by inhalation, or intravenously. In certain preferred embodiments, the composition is administered by subcutaneous injection.

### Dosing

The compositions disclosed herein may comprise about 10 μg to about 1 gram of hepcidin to the subject, such as about 100 μg to about 100 mg, about 200 μg to about 50 mg, or about 500 μg to about 10 mg, about 500 μg to about 5 mg, or about 500 μg to about 2 mg of hepcidin per dose. The compositions may comprise about 100 μg, about 150 μg, about 200 μg, about 250 μg, about 300 μg, about 333 μg, about 400 μg, about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin per dose.

Provided herein are methods of administering composition disclosed herein. Administering a composition of the invention comprising hepcidin to the subject may comprise administering a bolus, a depot, a sustained release formulation, a continuous infusion, multiple, and/or sequential doses of the composition.

The method may comprise administering the composition to the subject at least once per month, such as at least once per week. The method may comprise administering the composition to the subject 1, 2, 3, 4, 5, 6, or 7 times per week. In preferred embodiments, the method comprises administering the composition to the subject 1, 2, or 3 times per week.

The method may comprise administering about 10 μg to about 1 gram of hepcidin to the subject each time the composition is administered, such as about 100 μg to about 100 mg, about 200 μg to about 50 mg, about 500 μg to about 10 mg, about 500 μg to about 5 mg, or about 500 μg to about 2 mg of hepcidin or mini-hepcidin. The method may comprise administering about 100 μg, about 150 μg, about 200 μg, about 250 μg, about 300 μg, about 333 μg, about 400 μg, about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin to the subject each time the composition is administered.

In some embodiments, less than about 200 mg hepcidin is administered to a human subject each time the composition is administered. In some embodiments, less than about 150 mg hepcidin is administered to a human subject each time the composition is administered, such as less than about 100 mg, less than about 90 mg, less than about 80 mg, less than about 70 mg, less than about 60 mg, or less than about 50 mg. In some embodiments, less than 10 mg of hepcidin is administered to a human subject each time the composition is administered, such as less than about 9 mg, less than about 8 mg, less than about 7 mg, less than about 6 mg, less than about 5 mg, less than about 4 mg, less than about 3 mg, less than about 2 mg, or less than about 1 mg. In some embodiments, about 100 μg to about 10 mg of hepcidin is administered to a human subject each time the composition is administered, such as about 100 μg to about 9 mg, about 100 μg to about 8 mg, about 100 μg to about 7 mg, about 100 μg to about 6 mg, about 100 μg to about 5 mg, about 100 μg to about 4 mg, about 100 μg to about 3 mg, about 100 μg to about 2 mg, or about 100 μg to about 1 mg.

The invention will be more readily understood by reference to the following examples, which are included merely to illustrate certain aspects and embodiments of the present invention and are not intended to limit the invention.

### EXAMPLES

### Example 1: Initial Formulation Stability Studies

Initial hepcidin stability assays evaluated multiple concentrations (50, 100 and 150 mg/mL) of hepcidin-25 for stability at 5° C. over 4.5 months. Samples were analyzed by reversed-phase high-performance liquid chromatography (RP-HPLC). The primary degradation products observed were mono or di-oxidation of the methionine at position 21 in the hepcidin-25 sequence. Based on these results hepcidin-25 was formulated either in saline (40 mg/mL) or in saline (40 mg/mL) with methionine as an excipient (ten-fold molar excess relative to peptide concentration) to prevent oxidation of the peptide and placed on accelerated stability (37° C. and 45° C.). Samples were evaluated over 4.5 months and analyzed by RP-HPLC. Under all conditions, the rate of oxidation observed was slow. The addition of methionine as an excipient reduced the amount of oxidation even further.

### Example 2: Mechanism of Aggregate Formation

Hepcidin contains 8 cysteines, forming 4 disulfide bridges, each with the potential to become unstable under certain conditions. Accelerated stability studies (e.g. at 40° C.) and real time stability studies (e.g., at 5° C.), showed aggregation as the main mode of degradation as measured by size exclusion chromatography (SEC). Significant work was completed to address this degradation pathway.

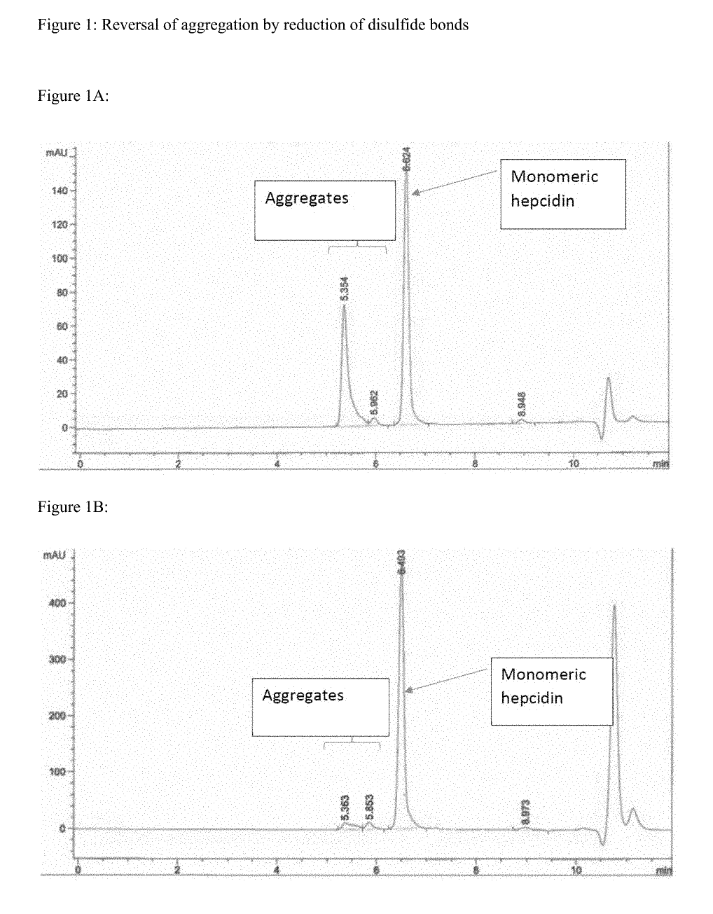

Briefly, the effect of potential intermolecular disulfide bonding on aggregation was studied by adding a reducing agent to aggregated material to break all disulfide bonds. Hepcidin-25 with an aggregate content of 35% as measured by SEC was used for this study. An aliquot of this material was treated with 20 mM Dithiothreitol (DTT) and samples were evaluated by SEC. As indicated in FIG. 1, aggregation is reversed upon breakage of all disulfide bonds.

Based on these results, the mechanism of aggregate formation was inferred to be due to intermolecular disulfide bonds between molecules of hepcidin. Aggregate formation is thus mediated by the breaking of both intra- and intermolecular disulfide bonds resulting in the formation of free sulfhydryl groups which can then react with other disulfide bonds (to form new disulfide bonds).

### Example 3: Effect of Thiol

In view of the role played by intermolecular disulfide bond formation in aggregate formation, and the contribution of thiol groups to that end, multiple sources of hepcidin-25 were analyzed for thiol using a thiol content assay and evaluated for stability. Commercially available hepcidin was used in these Examples, which while “purified” to contain only hepcidin, contained thiols and aggregates, and thus may be referred to as “unpurified” to distinguish it from the compositions of the invention. One lot with significant thiol was purified to demonstrate the effect of thiol on stability. For the purpose of this experiment the thiol impurity was removed as follows: Thiols were quenched using 1 molar equivalents of N-methylmaleimide (NEM). The solution was then purified by tangential flow filtration (TFF) using Millipore Pellicon 2 10 kDa cassettes for aggregate removal and Pall T-Series 1 kDa for ultra-filtration-diafiltration. The starting material was diluted to less than or equal to 0.1 mg/mL prior to processing through the 10 kDa system. The diluent consisted of 15% Acetonitrile/0.05% Trifluoracetic acid (TFA).

The initial unpurified material and the resulting purified material were formulated in 0.9% saline at either pH 2.5 or pH 5.6 and assessed for stability at 40° C. Samples were taken over 10 days and evaluated by SEC.

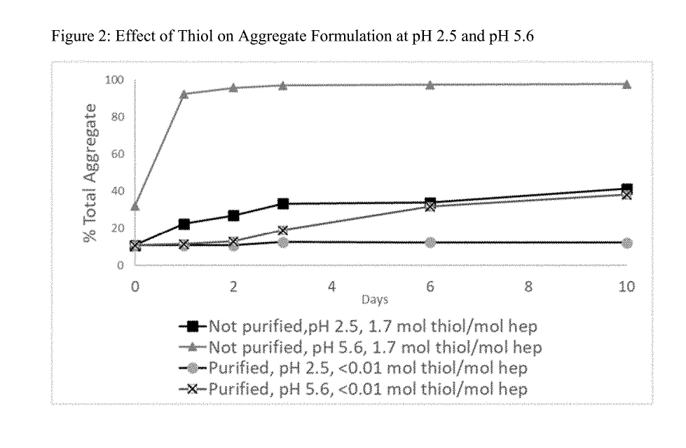

As indicated in Table 2, all batches of hepcidin analyzed pre-purification contained detectable levels of thiols and varying degrees of instability. Further purification of one lot of hepcidin containing significant levels of thiol resulted in material with thiol content less than the assay's limit of detection. Formulation of the purified hepcidin demonstrated increased stability in comparison to non-purified hepcidin formulations. FIG. 2 shows the effect of decreased thiol on aggregated formation at two different pH values.

TABLE 2
Thiol content of various batches of hepcidin-25
before and after purification

| | Average mole thiol/ | |
|---|---|---|
| Hepcidin Source | [mole hepcidin] | Stability |
| Vendor 1, Lot 1 | 0.17 | Unstable, rapid |
| | | aggregate formation |
| Vendor 1, Lot 2 | >0.2 | Unstable, rapid |
| | | aggregate formation |
| Vendor 2, Lot 1 | 0.03 | Unstable, slower |
| | | aggregate formation |
| Vendor 2, Lot 2 | 0.03 | Unstable, slower |
| | | aggregate formation |
| Vendor 2, Lot 2 | 0.02 | Unstable, slower |
| | | aggregate formation |
| Vendor 1, Lot 1 purified | <0.01 | Stable, very slow |
| | | aggregate formation |
| Vendor 3, Lot 1 | 0.03 | Unstable, slower |
| | | aggregate formation |

Lower quantifiable Limit = 0.01 mole thiol/mole hepcidin

As indicated in FIG. 2, the unpurified sample (with 1.7 mole thiol/mole hepcidin) immediately starts to aggregate at pH 5.6 (reaching 38% within time it took to test the material by SEC) and almost fully aggregates (>95%) after 24 hours, while the low pH sample (pH 2.5) aggregates more slowly. For the purified sample, with no detectable thiol, aggregation is substantially slower at pH 5.6, only reaching about 38% after 10 days at 40° C. The low pH sample (pH 2.5) showed no aggregation over the same period. The addition of thiol scavenger significantly reduces the amount of aggregate formation.

### Example 4: Effect of pH on Aggregate Formation

Aggregate formation was also significantly influenced by pH. As illustrated in the previous example (FIG. 2), even unpurified hepcidin (containing thiol), shows substantially increased stability at a very low pH. Conversely, purified hepcidin at a higher pH exhibited comparable stability to the unpurified hepcidin at low pH, thus showing a strong pH dependence.

Based on the significant differences observed when comparing samples at pH 2.5 and pH 5.6, smaller changes in pH (e.g., pH 4.2 vs pH 4.8) were evaluated.

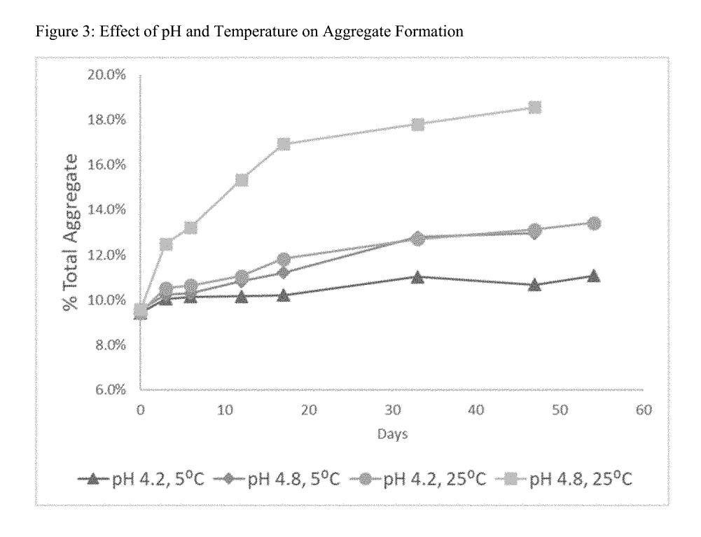

Hepcidin-25, with 9% starting aggregate and purified to remove thiol, was dissolved in 0.9% sodium chloride and adjusted to either pH 4.2 or pH 4.8. Samples were filtered using a 0.2 μm syringe filter and aliquoted into glass screw cap amber vials. Samples were assessed for stability at both 5° C. and 25° C. Samples were taken over about 50 days and evaluated by visual inspection, pH and SEC. As indicated in FIG. 3 the effect of pH 4.2 versus 4.8 was also significant, with pH 4.2 being more stable than pH 4.8.

### Example 5: Effect of Initial Aggregate Level on Future Aggregate Formation

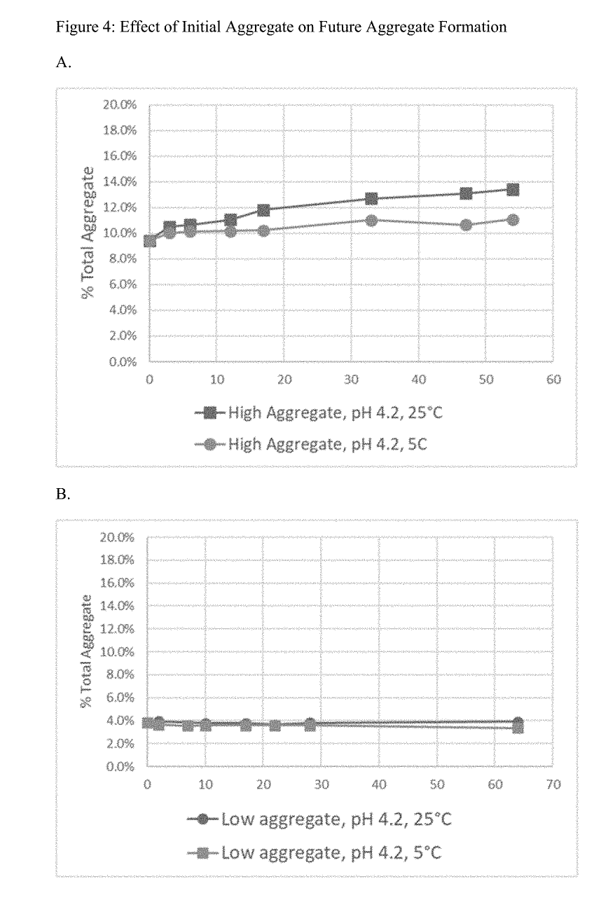

Because aggregate is thermodynamically unstable, initial aggregate level influences future total aggregate formation. FIG. 4 shows the effect of 9% starting aggregate versus 4% starting aggregate at both room temperature and 5° C. where the hepcidin has been purified to remove thiol.

Samples from lots of high and low initial aggregate levels were prepared at 20 mg/mL and pH 4.2 in 0.9% saline. Samples were filtered using a 0.2 μm syringe filter and aliquoted into glass screw cap amber vials. Stability at 5° C. and 25° C. was assessed with readings taken over about 56 days, i.e., evaluated by visual inspection, pH and SEC.

As indicated in FIG. 4, a higher initial aggregation increases the rate of future aggregation.

### Example 6: Use of Thiol Scavengers to Prevent Oxidation

Since aggregation was observed to be primarily driven by disruption of intramolecular disulfide bonding and promotion of intermolecular disulfide bond formation, propagated by thiol, a thiol scavenger, such as maleic acid, was evaluated.

Maleic acid was added to solutions of hepcidin-25 (20 mg/mL), at concentrations of each of 1, 2 and 4 mg/mL maleic acid (corresponding to a hepcidin to maleic acid ratio of 0.83:1, 0.42:1 and 0.21:1 respectively), and at a pH of 4.8. These solutions were filtered using a 0.2 μm syringe filter and aliquoted into glass screw cap amber vials. Stability was assessed at 5° C. and 25° C., with samples taken over the course of 47 days for evaluation by visual inspection, pH and SEC.

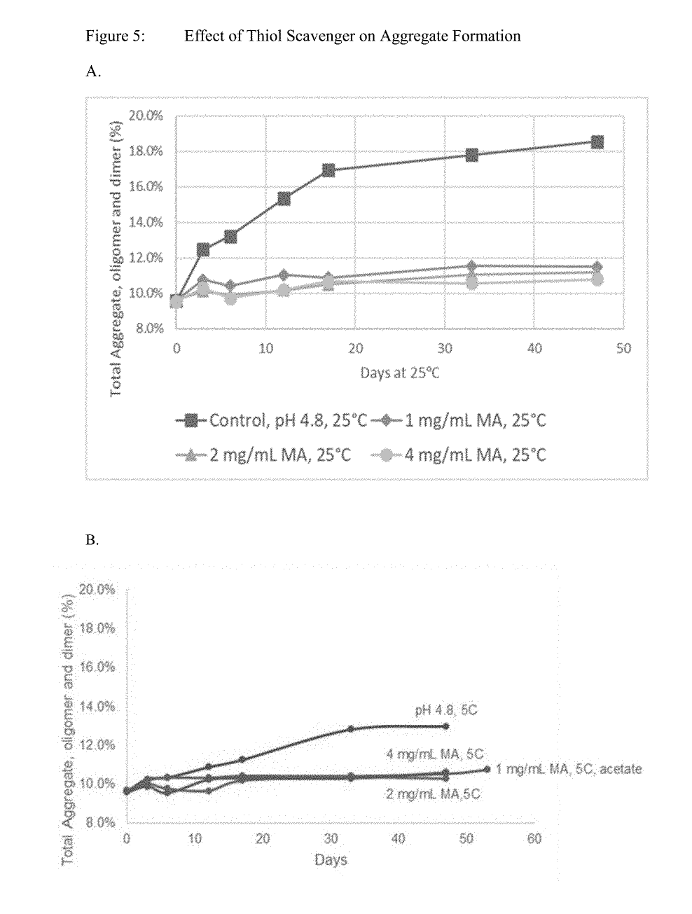

As indicated in FIG. 5, all three concentrations evaluated imparted significant improvement in stability when compared to maleic acid-free control at 2-8° C. and 25° C.

### Example 7: Effect of Salt Form

Initially, hepcidin-25 was synthesized as the TFA salt and required addition of base to increase pH. Since the disulfides in hepcidin are sensitive to base, it was hypothesized that addition of base might introduce additional instability by disruption of some small number of disulfide bonds (due to transient localization of excessively high pH as base is added to solution). Accordingly, a study was conducted to evaluate other salt forms including acetate and chloride.

The trifluoroacetate, chloride and acetate salts of hepcidin were prepared and all three salt forms were formulated in 0.9% saline at pH 4.2. The hepcidin concentration was 20 mg/mL with a maleic acid concentration of either 0 mg/mL or 1 mg/mL. Samples were filtered using a 0.2 μm syringe filter and aliquoted into glass screw cap amber vials. The samples were assessed for stability at 5° C. and 25° C. Over the course of 47 days the samples were evaluated by visual inspection, pH and SEC.

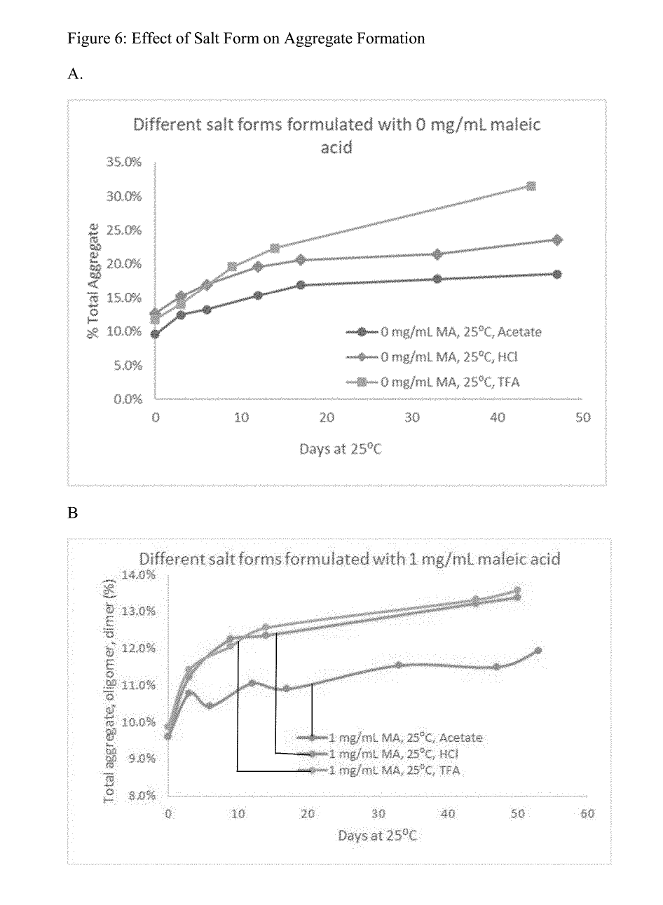

When preparing solutions of hepcidin, the acetate salt form gives a native pH of about 5 and thus requires acidification to adjust pH to 4.2; in contrast, the HCl salt gives a native pH of about 2.5 and thus requires the addition of base. As indicated in FIG. 6, the acetate salt form appears to be the most stable of the three salt forms under these conditions. When no maleic acid is present, adjustment of the pH leads to a slight increase in initial aggregate and potentially faster aggregate formation over time. When maleic acid is present, there is an initial increase in aggregation for the TFA and HCl salt forms, but then continued aggregation formation is slowed by the presence of maleic acid. However, while acetate is preferred, other salt forms (including chloride and TFA) are still useful as pH adjustment could be done more slowly to prevent the initial instability observed in this study.

### Example 8: Stability Evaluation of Hepcidin Formulations

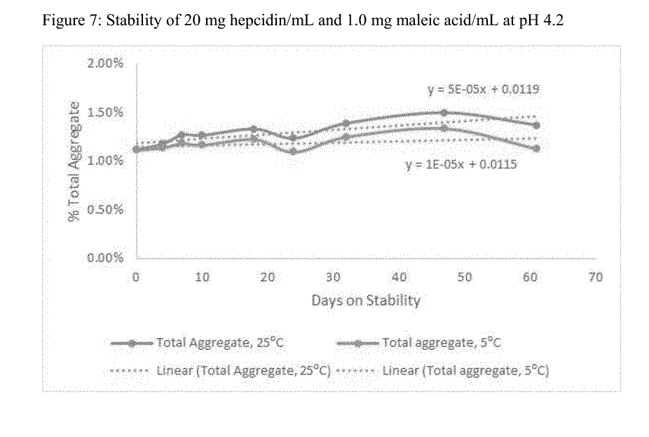

Based on the results of the preceding experiments, stability over time at 5° C. and 25° C. of two formulations was evaluated. The first formulation comprised 20 mg/mL hepcidin acetate salt and 1 mg/mL maleic acid (hepcidin to maleic acid molar ratio=0.8:1) at pH 4.2. As illustrated in FIG. 7, the hepcidin formulation was stable with respect to aggregate formation. Linear regression analysis for each condition predicted a level of aggregation that increased no more than 0.02% or 2 years at 5° C. and no more than 0.05% over 2 years at 25° C.

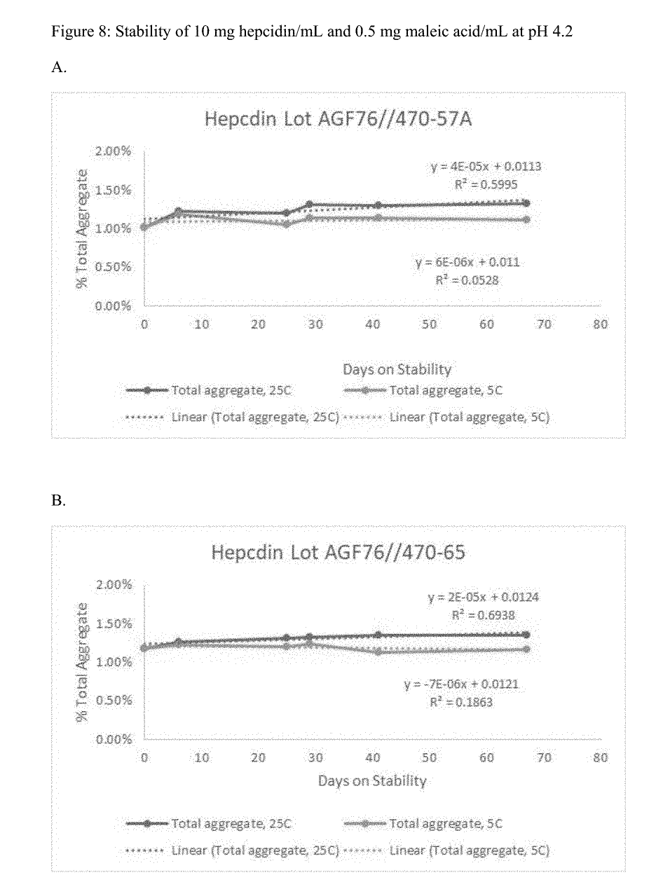

The second formulation comprised the acetate salt of hepcidin formulated at concentration of 10 mg/mL and 0.5 mg/mL maleic acid (concentration ratio of 20:1 (molar ratio of 0.8:1). Samples were assessed for stability at 5° C. and 25° C. over the course of 66 days. As summarized in FIG. 8, the results indicated that the second formulation of hepcidin was stable with respect to aggregation. Similarly, linear regression analysis for each condition provided an equation that predicted the level of aggregation would increase no more than 0.02% over 2 years at 5° C. and no more than 0.04% over 2 years at 25° C.

### Example 9: Superior Clinical Utility of an Exemplary Formulation Vs. Unpurified, Unformulated Hepcidin

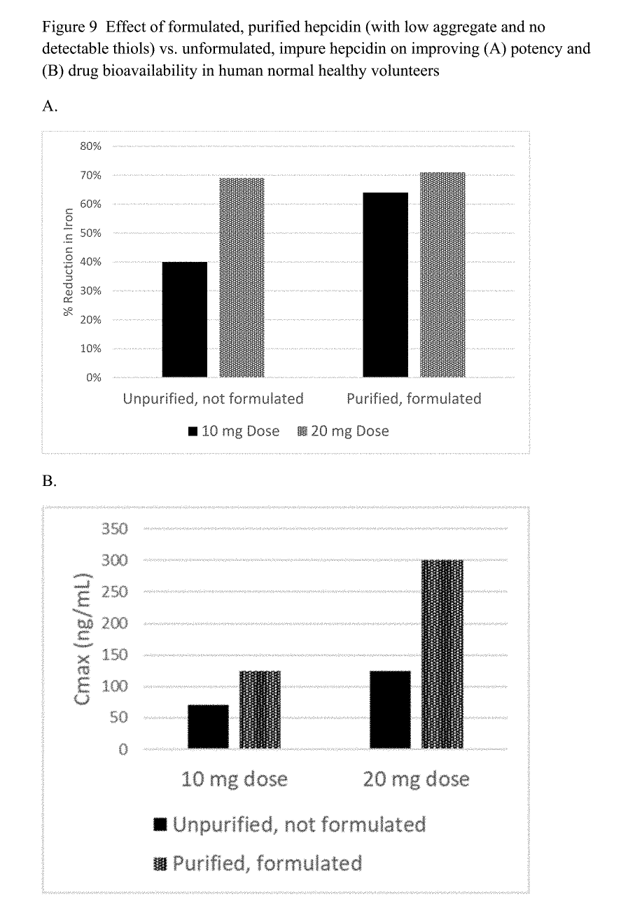

Clinical studies demonstrate the advantage of an exemplary formulation of purified hepcidin vs. a formulation of unpurified hepcidin. In a first clinical study, hepcidin containing thiol and about 12% aggregate formulated in normal saline at pH 4.6 was tested in healthy volunteers. In a second clinical study, an exemplary formulation containing purified hepcidin with no detectable thiol and less than 0.5% aggregation at pH 4.2 with maleic acid at molar ratio of about 1:1 was tested in healthy volunteers. FIGS. 9A and 9B show the improved efficacy of the exemplary formulation vs the impure hepcidin solution. As indicated in FIG. 9A, the iron lowering effect (as measured by serum iron) was significantly better in the exemplary formulation. It required about 2 times as much of the unpurified hepcidin in saline to achieve the same reduction in serum iron. As indicated in FIG. 9B, the amount of drug absorbed was also significantly improved in the purified, formulated hepcidin.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control. While specific aspects of the patient matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

### ADDITIONAL EMBODIMENTS

### Embodiment P1

A composition comprising a purified hepcidin, wherein the composition is substantially free of thiol.

### Embodiment P2

The composition of embodiment P1, wherein the thiol content is less than or equal to 0.2 mole thiol/mole hepcidin.

### Embodiment P3

The composition of embodiment P1, wherein the thiol content is less than or equal to 0.1 mole thiol/mole hepcidin.

### Embodiment P4

The composition of embodiment P1, wherein the thiol content is less than or equal to 0.05 mole thiol/mole hepcidin.

### Embodiment P5

The composition of any one of embodiments P1 to P4, wherein the purified hepcidin is substantially free of aggregated hepcidin.

### Embodiment P6

A composition comprising a purified hepcidin, wherein the purified hepcidin is substantially free of aggregated hepcidin.

### Embodiment P7

The composition of any one of embodiments P5 or P6, wherein less than 10% of the hepcidin is aggregated.

### Embodiment P8

The composition of any one of embodiments P5 or P6, wherein less than 5% of the hepcidin is aggregated.

### Embodiment P9

The composition of any one of embodiments P5 or P6, wherein less than 3% of the hepcidin is aggregated.

### Embodiment P10

The composition of any one of embodiments P1 to P9, wherein the purified hepcidin is a salt.

### Embodiment P11

The composition of embodiment P10, wherein the purified hepcidin is a salt with an anionic counterion.

### Embodiment P12

The composition of embodiment P11, wherein the anionic counter ion comprises at least one of acetate, trifluoroacetate, chloride, bromide, citrate, sulfate, borate, lactate, maleate, malate, fumarate, phosphate, diphosphate, gluconate, uronate, succinate, propionate, tartrate, nitrate, mesylate, calcium, potassium or a mixture thereof.

### Embodiment P13

The composition of any one of embodiments P1 to P12, further comprising a thiol scavenger.

### Embodiment P14

The composition of embodiment P13, wherein the thiol scavenger comprises at least one of an N-alkylmaleimide, iodine, maleic acid, sodium maleate, fumaric acid, salts thereof, or esters thereof.

### Embodiment P15

The composition of embodiment P14, wherein the thiol scavenger is N-ethylmaleimide (NEM).

### Embodiment P16

The composition of embodiment P14, wherein the thiol scavenger is maleic acid.

### Embodiment P17

The composition of any one of embodiments P13 to P16, wherein the molar ratio of purified hepcidin to thiol scavenger is from about 0.5:1 to about 10:1.

### Embodiment P18

The composition of embodiment P17, wherein the molar ratio of purified hepcidin to thiol scavenger is 1:1.

### Embodiment P19

The composition of any one of the preceding embodiments, wherein the composition is an aqueous solution.

### Embodiment P20

The composition of embodiment P19, wherein the pH of the composition is between pH 2 and pH 6.

### Embodiment P21

The composition of embodiment P19, wherein the pH of the composition is between pH 2 and pH 5.

### Embodiment P22

The composition of any one of embodiments P19 to P21, wherein the composition is substantially free of aggregated hepcidin.

### Embodiment P23

The composition of any one of embodiments P19 to P22, wherein the aqueous solution further comprises sodium chloride.

### Embodiment P24

The composition of embodiment P23, wherein the aqueous solution is normal saline.

### Embodiment P25

The composition of any one of embodiments P1 to P24, wherein the concentration of hepcidin is from about 0.1 mg/mL to about 40 mg/mL.

### Embodiment P26

The composition of embodiment P25, wherein the concentration of hepcidin is at least 0.5 mg/mL.

### Embodiment P27

The composition of embodiment P25, wherein the concentration of hepcidin is at least 1 mg/mL.

### Embodiment P28

The composition of embodiment P25, wherein the concentration of hepcidin is at least 5 mg/mL.

### Embodiment P29

The composition of embodiment P25, wherein the concentration of hepcidin is at least 10 mg/mL.

### Embodiment P30

The composition of embodiment P25, wherein the concentration of hepcidin is at least 20 mg/mL.

### Embodiment P31

The composition of embodiment P25, wherein the concentration of hepcidin is at least 30 mg/mL.

### Embodiment P32

The composition of embodiment P25, wherein the concentration of hepcidin is at least 40 mg/mL.

### Embodiment 33

The composition of any one of embodiments P1 to P32 wherein the hepcidin comprises human hepcidin.

### Embodiment P34

The composition of embodiment P33, wherein the hepcidin comprises an amino acid sequence of SEQ ID NO: 1.

### Embodiment P35

The composition of embodiment P33, wherein the hepcidin comprises an amino acid sequence of SEQ ID NO: 2.

### Embodiment P36

The composition of embodiment P33, wherein the hepcidin comprises an amino acid sequence of SEQ ID NO: 3.

### Embodiment P37

The composition of embodiment P33, wherein the hepcidin comprises an amino acid sequence of SEQ ID NO: 4.

### Embodiment P38

The composition of embodiment P33, wherein the hepcidin comprises an amino acid sequence of SEQ ID NO: 5.

### Embodiment P39

The composition of embodiment P33, wherein the hepcidin comprises an amino acid sequence of SEQ ID NO: 6.

### Embodiment P40

The composition of embodiment P33, wherein the hepcidin comprises an amino acid sequence of SEQ ID NO: 7.

### Embodiment P41

The composition of embodiment P33, wherein the hepcidin comprises an amino acid sequence of SEQ ID NO: 8.

### Embodiment P42

The composition of embodiment P33, wherein the hepcidin comprises an amino acid sequence of SEQ ID NO: 9.

### Embodiment P43

The composition of embodiment P33, wherein the hepcidin comprises an amino acid sequence of SEQ ID NO: 10.

### Embodiment P44

The composition of embodiment P33, wherein the hepcidin is the prepropeptide form.

### Embodiment P45

The pharmaceutical composition of embodiment P33, wherein the hepcidin is the propeptide form.

### Embodiment P46

A pharmaceutical composition comprising at least 5 mg/mL hepcidin, or a pharmaceutically acceptable salt thereof, in an aqueous solution having a pH ranging from about 4.0 to about 4.5, wherein the aqueous solution further comprises a thiol scavenger.

### Embodiment P47

The pharmaceutical composition of embodiment P46, wherein the molar ratio of hepcidin, or a pharmaceutically compatible acceptable salt thereof, to the thiol scavenger is 1:1.

### Embodiment P48

The pharmaceutical composition of any one of embodiments P46 or P47, wherein the thiol scavenger is maleic acid.

### Embodiment P49

The pharmaceutical composition of any one of embodiments P46 to P48, wherein the composition is substantially free of thiol.

### Embodiment P50

The pharmaceutical composition of embodiment P49, wherein the aqueous solution further comprises sodium chloride.

### Embodiment P51

The pharmaceutical composition of embodiment P49, wherein the aqueous solution is normal saline.

### Embodiment P52

The pharmaceutical composition of any one of embodiments P49 to P51, wherein less than 3% of hepcidin is aggregated.

### Embodiment P53

The pharmaceutical composition of any one of embodiments P49 to P51, wherein less than 2% of hepcidin is aggregated.

### Embodiment P54

The pharmaceutical composition of any one of embodiments P49 to P51, wherein the hepcidin comprises human hepcidin.

### Embodiment P55

The pharmaceutical composition of embodiment P54, wherein the hepcidin comprises at least one of the amino acid sequences set forth in SEQ ID NOs: 1 through 10.

### Embodiment P56

The pharmaceutical composition of any one of embodiments P46 to P55, wherein the hepcidin is suspended in the composition.

### Embodiment P57

The pharmaceutical composition of embodiment 56, wherein the hepcidin is a colloid in the composition.

### Embodiment P58

The pharmaceutical composition of any one of embodiments P46 to P56, wherein the hepcidin is dissolved in the composition.

### Embodiment P59

The composition of any one of embodiments P1 to P58, wherein the composition is formulated for parenteral administration.

### Embodiment P60

The composition of any one of embodiments P1 to P58, wherein the composition is formulated for intravenous administration.

### Embodiment P61

The composition of any one of embodiments P1 to P58, wherein the composition is formulated for subcutaneous administration.

### Embodiment P62

The composition of any one of embodiments P1 to P58, wherein the composition is formulated for intramuscular administration.

### Embodiment P63

The composition of any one of embodiments P1 to P58, wherein the composition is formulated for intranasal administration.

### Embodiment P64

The composition of any one of embodiments P1 to P58, wherein the composition is formulated for oral administration.

### Embodiment P65

The composition of any one of embodiments P1-P64, wherein the composition is at least 25% more potent upon administration to a subject than the same amount of a comparable hepcidin composition having, prior to administration, at least one of i) greater than 3% of the hepcidin in aggregated form or ii) an average thiol content of greater than 0.015 mole thiol per mole hepcidin.

### Embodiment P66

The composition of any one of embodiments P1-P64, wherein the composition results in at least 25% greater iron reduction upon administration to a subject than the same amount of a comparable hepcidin composition having, prior to administration, at least one of i) greater than 3% of the hepcidin in aggregated form or ii) an average thiol content of greater than 0.015 mole thiol per mole hepcidin.

### Embodiment P67

The composition of any one of embodiments P1-P64, wherein the composition has at least 25% longer duration of effect upon administration to a subject than the same amount of a comparable hepcidin composition having, prior to administration, at least one of i) greater than 3% of the hepcidin in aggregated form ii) an average thiol content of greater than 0.015 mole thiol per mole hepcidin.

### Embodiment P68

A method of treating or preventing a condition in a subject, comprising administering the composition of any one of embodiments P1 to P64.

### Embodiment P69

The method of embodiment P68, wherein the condition is α-thalassemia, thalassemia intermedia, β-thalassemia, hemochromatosis, sickle cell disease, anemia, refractory anemia, hemolytic anemia, hepatocarcinoma, cardiomyopathy, diabetes, a viral infection, a bacterial infection, a fungal infection, or a protist infection.

### Embodiment P70

The method of embodiment P69, wherein the condition is α-thalassemia.

### Embodiment P71

The method of embodiment P69, wherein the condition is thalassemia intermedia.

### Embodiment P72

The method of embodiment P69, wherein the condition is β-thalassemia.

### Embodiment P73

The method of embodiment P69, wherein the condition is sickle cell disease.

### Embodiment P74

The method of embodiment P69, wherein the condition is Chagas disease, malaria, African sleeping sickness, or leishmaniasis.

### Embodiment P75

The method of embodiment P69, wherein the condition is malaria.

### Embodiment P76

The method of embodiment P69, wherein the condition is a viral infection, and the virus is hepatitis B, hepatitis C, or dengue virus.

### Embodiment P77

The method of embodiment P69, wherein the condition is a bacterial infection and the bacterial infection is tuberculosis.

### Embodiment P78

The composition of any one of embodiments P1-P64, wherein when the composition is stored at 5° C. for at least 2 years, the amount of hepcidin aggregates in the composition is less than 3%.

### Embodiment P79

The composition of any one of embodiments P1-P64, wherein when the composition is stored at 25° C. for at least 2 years, the amount of hepcidin aggregates in the composition is less than 5%.

1. A composition comprising a purified hepcidin, wherein the composition is substantially free of thiol.

2. The composition of claim 1, wherein the average thiol content is less than or equal to 0.2 mole thiol/mole hepcidin.

3-4. (canceled)

5. The composition of claim 1, wherein the purified hepcidin is substantially free of aggregated hepcidin.

6. (canceled)

7. The composition of claim 1, wherein less than 10% of the hepcidin is aggregated.

8-9. (canceled)

10. The composition of claim 1, wherein the purified hepcidin is a salt with an anionic counterion.

11. The composition of claim 10, wherein the anionic counter ion comprises at least one of acetate, trifluoroacetate, chloride, bromide, citrate, sulfate, borate, lactate, maleate, malate, fumarate, phosphate, diphosphate, gluconate, uronate, succinate, propionate, tartrate, nitrate, mesylate, calcium, potassium or a mixture thereof.

12. The composition of claim 1, further comprising a thiol scavenger.

13. The composition of claim 12, wherein the thiol scavenger comprises at least one of an N-alkylmaleimide (NEM), iodine, maleic acid, sodium maleate, fumaric acid, salts thereof, or esters thereof.

14. (canceled)

15. The composition of claim 12, wherein the molar ratio of purified hepcidin to thiol scavenger is from about 0.20:1 to about 10:1.

16. (canceled)

17. The composition of claim 1, wherein the composition is an aqueous solution.

18. The composition of claim 17, wherein the pH of the composition is between pH 2 and pH 6.

19-22. (canceled)

23. The composition of claim 17, wherein the concentration of hepcidin is from about 0.1 mg/mL to about 40 mg/mL.

24-30. (canceled)

31. The composition of claim 1 wherein the hepcidin comprises human hepcidin.

32. The composition of claim 31, wherein the hepcidin comprises an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

33. (canceled)

34. A pharmaceutical composition comprising at least 5 mg/mL hepcidin, or a pharmaceutically acceptable salt thereof, in an aqueous solution having a pH ranging from about 4.0 to about 4.5, wherein the aqueous solution further comprises a thiol scavenger.

35. The pharmaceutical composition of claim 34, wherein the molar ratio of hepcidin, or a pharmaceutically compatible acceptable salt thereof, to the thiol scavenger is 1:1.

36. The pharmaceutical composition of claim 34, wherein the thiol scavenger is N-alkylmaleimide (NEM), iodine, maleic acid, sodium maleate, fumaric acid, salts thereof, or esters thereof.

37. The pharmaceutical composition of claim 34, wherein the composition is substantially free of thiol.

38. (canceled)

39. The pharmaceutical composition of claim 37, wherein less than 3% of hepcidin is aggregated.

40. The pharmaceutical composition of claim 37, wherein the hepcidin comprises human hepcidin or synthetic hepcidin.

41. The pharmaceutical composition of claim 40, wherein the hepcidin comprises at least one of the amino acid sequences set forth in SEQ ID NOs: 1 through 10.

42-43. (canceled)

44. The composition of claim 1, wherein the composition is at least 25% more potent upon administration to a subject than the same amount of a comparable hepcidin composition having, prior to administration, at least one of i) greater than 3% of the hepcidin in aggregated form or ii) an average thiol content of greater than 0.015 mole thiol per mole hepcidin.

45. The composition of claim 1, wherein the composition results in at least 25% greater iron reduction upon administration to a subject than the same amount of a comparable hepcidin composition having, prior to administration, at least one of i) greater than 3% of the hepcidin in aggregated form or ii) an average thiol content of greater than 0.015 mole thiol per mole hepcidin.

46. The composition of claim 1, wherein the composition has at least 25% longer duration of effect upon administration to a subject than the same amount of a comparable hepcidin composition having, prior to administration, at least one of i) greater than 3% of the hepcidin in aggregated form ii) an average thiol content of greater than 0.015 mole thiol per mole hepcidin.

47-51. (canceled)


SEQUENCE LISTING (10 sequences)

SEQ ID NO: 1 - protein, 25 aa, Homo sapiens
DTHFPICIFCCGCCHRSKCGMCCKT

SEQ ID NO: 2 - protein, 21 aa, Homo sapiens
PICIFCCGCCHRSKCGMCCKT

SEQ ID NO: 3 - protein, 19 aa, Homo sapiens
PICIFCCGCCHRSKCGMCC

SEQ ID NO: 4 - protein, 20 aa, Homo sapiens
ICIFCCGCCHRSKCGMCCKT

SEQ ID NO: 5 - protein, 17 aa, Homo sapiens
CIFCCGCCHRSKCGMCC

SEQ ID NO: 6 - protein, 25 aa, Artificial Sequence
  Description of Artificial Sequence Synthetic peptide
  MOD_RES (1)..(2): Any amino acid
  MOD_RES (4)..(4): Any amino acid
  MOD_RES (6)..(6): Any amino acid
  MOD_RES (8)..(9): Any amino acid
  MOD_RES (24)..(25): Any amino acid
XXHXPXCXXCCGCCHRSKCGMCCXX

SEQ ID NO: 7 - protein, 21 aa, Artificial Sequence
  Description of Artificial Sequence Synthetic peptide
  MOD_RES (2)..(2): Any amino acid
  MOD_RES (4)..(5): Any amino acid
  MOD_RES (21)..(21): Any amino acid
PXCXXCCGCCHRSKCGMCCKX

SEQ ID NO: 8 - protein, 19 aa, Artificial Sequence
  Description of Artificial Sequence Synthetic peptide
  MOD_RES (2)..(2): Any amino acid
  MOD_RES (4)..(5): Any amino acid
PXCXXCCGCCHRSKCGMCC

SEQ ID NO: 9 - protein, 20 aa, Artificial Sequence
  Description of Artificial Sequence Synthetic peptide
  MOD_RES (1)..(1): Any amino acid
  MOD_RES (3)..(4): Any amino acid
  MOD_RES (12)..(13): Any amino acid
  MOD_RES (16)..(16): Any amino acid
  MOD_RES (20)..(20): Any amino acid
XCXXCCGCCHRXXCGXCCKX

SEQ ID NO: 10 - protein, 17 aa, Artificial Sequence
  Description of Artificial Sequence Synthetic peptide
  MOD_RES (2)..(3): Any amino acid
  MOD_RES (11)..(12): Any amino acid
  MOD_RES (15)..(15): Any amino acid
CXXCCGCCHRXXCGXCC